(19) United States
(12) Patent Application Publication
(10) Pub. No.: US 2015/0038575 A1
(43) Pub. Date: Feb. 5, 2015

(54) MELANOGENESIS-INHIBITORY CONSTITUENTS OF Ligusticum sinense

(71) Applicant: Ching Kuo LEE, New Taipei City (TW)

(72) Inventors: Ching-Kuo LEE, New Taipei City (TW); Yi-Tzu CHU, Taipei City (TW); Horng-Ji LAI, Taipei City (TW); Wen-Li LIANG, New Taipei City (TW)

(21) Appl. No.: 14/447,601

(22) Filed: Jul. 30, 2014

(30) Foreign Application Priority Data
Aug. 1, 2013 (TW) 102127636

(51) Int. Cl. C07D 307/88; C07C 59/64

(52) U.S. Cl. CPC C07D 307/88; C07C 59/64
USPC 514/470; 562/468; 514/570

(57) ABSTRACT

Melanogenesis-inhibitory constituents of Ligusticum sinense are disclosed. The melanogenesis-inhibitory constituents can be compound I, II, or III having the chemical structures as follow, respectively.

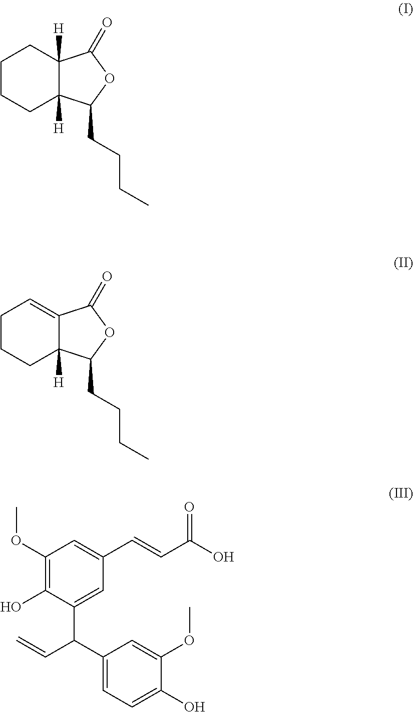 [SMILES: CCCC[C@@H]1OC(=O)[C@H]2CCCC[C@@H]12 ; CCCC[C@@H]1OC(=O)C2=CCCC[C@H]21 ; C=CC(c1ccc(O)c(OC)c1)c1cc(/C=C/C(=O)O)cc(OC)c1O]

### RELATED APPLICATIONS

This application claims the priority benefit of Taiwan application serial no.

102127636, filed Aug. 1 2013, the full disclosure of which is incorporated herein by reference.

### BACKGROUND

1. Technical Field

The disclosure relates to melanogenesis-inhibitory constituents of Ligusticum sinense.

2. Description of Related Art

Melanin is primarily responsible for skin color, but also plays an important role in preventing sunburn. A physiological response of human skin will produce melanin to prevent sunburn when exposed to UV light. The melanogenesis is regulated by enzyme, such as tyrosinase and related proteins. Therefore, inhibition of the tyrosinase catalyzed steps of melanogenesis to reduce melanogenesis is the most common skin whitening method. Since most women want to avoid UV tanning in Asia, screening a compound that can inhibit tyrosinase or melanogenesis from natural resources would be useful.

### SUMMARY

In one aspect, the present invention is directed to an isolated compound III having a chemical structure as follow.

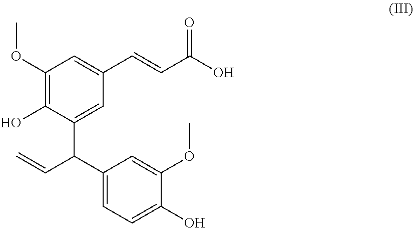 [SMILES: C=CC(c1ccc(O)c(OC)c1)c1cc(/C=C/C(=O)O)cc(OC)c1O]

In another aspect, the present invention is directed to a method of inhibiting melanogenesis. The method comprises a step of applying a composition comprising an effective amount of a compound having at least one of the chemical structures shown below on skin to inhibit melanogenesis.

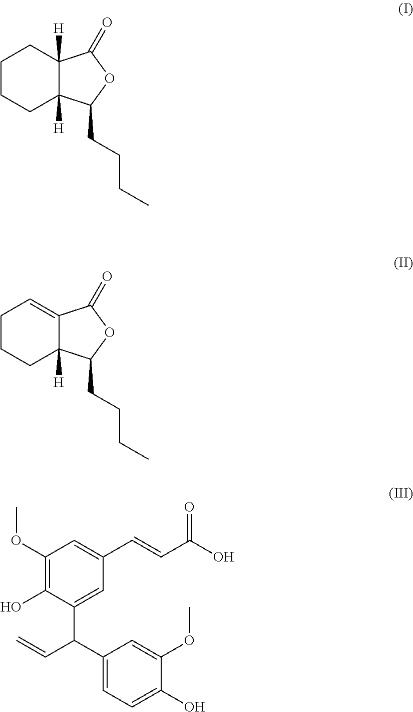 [SMILES: CCCC[C@@H]1OC(=O)[C@H]2CCCC[C@@H]12 ; CCCC[C@@H]1OC(=O)C2=CCCC[C@H]21 ; C=CC(c1ccc(O)c(OC)c1)c1cc(/C=C/C(=O)O)cc(OC)c1O]

The foregoing presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later. Many of the attendant features will be more readily appreciated as the same becomes better understood by reference to the following detailed description considered in connection with the accompanying drawings.

### BRIEF DESCRIPTION OF THE DRAWINGS

FIG. 1 is an ESI-MS spectrum of the compound III.

FIG. 2 is an IR spectrum of the compound III.

FIG. 3 is a ¹H NMR (500 MHz, acetone-d₆) spectrum of the compound III.

FIG. 4 is a ¹³C NMR (125 MHz, acetone-d₆) spectrum of the compound

FIG. 5 is a HSQC (acetone-d₆) spectrum of the compound III.

FIG. 6 is a HMBC (acetone-d₆) spectrum of the compound III.

FIG. 7 is a NOESY (acetone-d₆) spectrum of the compound III.

FIG. 8A is an experimental result of the cell viability of B16-F10 murine melanoma cells.

FIG. 8B is an experimental result of the melanin content in B16-F10 murine melanoma cells.

FIG. 8C is an experimental result of the cell viability of Human Epidermal Skin Equivalents (Leiden epidermal models; LEMs).

### DETAILED DESCRIPTION

Ligusticum sinense Oliver is a traditional Chinese medicine, and is also called as Chinese Lovage. In two thousand years ago, Chinese people used it to expel wind-cold, relieve pain, and whiten and moisturize skin. In the following experiment, a methanol extract of Ligusticum sinense Oliver is purified and identified. Then, the purified compounds from the methanol extract underwent assays of activity of inhibiting tyrosinase, cell viability of melanocytes, and melamine content in melanocytes to find compounds that can whiten skin.

### Preparation and Screening of Ligusticum sinense Oliver's Extract

9.9 kg rhizome of Ligusticum sinense Oliver was smashed, and 40 L of methanol was used to cold dip the powder of Ligusticum sinense Oliver to obtain an extract solution. The extracting process above was repeated for 3 times. After concentrated under a reduced pressure, 1758 g of a crude extract was obtained, and yield was 17.8%.

1458 g of the crude extract was partitioned by 1:1 (v/v) of water and ethyl acetate (EA) for 3 times. The EA extracted layer was collected. The weight of the obtained EA extracted layer was 415 g.

250 g of EA extracted layer was mixed with 375 g of 0.2 mm silica gel and absorbed by the silica gel. 3350 g of 40-63 μm silica gel was used to fill a column for preparing chromatography. Next, various volume ratios of n-hexane (Hex) and EA (Hex/EA=95/5, 90/10, 80/20, 60/40, 40/60, v/v) was used as eluent. Each volume ratio of eluent used was 20 L, and each 500 mL of the eluent was collected in one bottle. Each bottle was preliminarily analyzed by thin layer chromatography (TLC). The detailed related information above was listed in the Table 1 below.

TABLE 1
Solvent used to collect eleuent of column chromatography

| | | Solvent Volume Ratio |
|---|---|---|
| Parts | Collect Ranges | of Eluent |
| 1 | Bottles 1-39 (total 39 bottles) | 95/5 (v/v) Hex/EA |
| 2 | Bottles 40-77 (total 38 bottles) | 90/10 (v/v) Hex/EA |
| 3 | Bottles 78-115 (total 38 bottles) | 80/20(v/v) Hex/EA |
| 4 | Bottles 116-155 (total 40 bottles) | 60/40 (v/v) Hex/EA |
| 5 | Bottles 156-196 (total 41 bottles) | 40/60 (v/v) Hex/EA |

According to the analysis result of TLC, the total eluent was divided into 5 parts and then dried respectively. Next, high performance liquid chromatography (HPLC) was used to perform purification steps.

After performing the HPLC purification steps, 24 compounds were isolated, and three of them (compound I, II, Ill) have inhibitory activity of melanogenesis. The chemical structures and the HPLC purification conditions are listed Table 2 below. The compound I (Bartschat, D., Beck, T., & Mosandl, A. (1997) Stereoisomeric flavor compounds. 79. Simultaneous enantioselective analysis of 3-butylphthalide and 3-butylhexahydrophthaiide stereoisomers in celery, celeriac, and fennel. Journal of Agricultural and Food Chemistry, 45, 4551-4557) and the compound II (Oguro, D., & Watanabe, H. (2011) Synthesis and sensory evaluation of all stereoisomers of sedanolide. Tetrahedron, 67, 777-781) are known compounds, and the compound III is a new compound.

TABLE 2
The chemical structures and HPLC separation conditions of the
compounds I, II, and III.

| com- | | Separation conditions |
|---|---|---|
| pound | Chemical structure | of HPLC* |
| I | 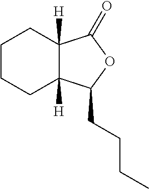 | Mobile phase: n-Hexane/EA = 96/4 Retention time: 23.5 min |
| II | 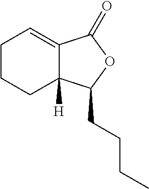 | Mobile phase: n-Hexane/EA = 95/5 Retention time: 19.3 min |
| III | 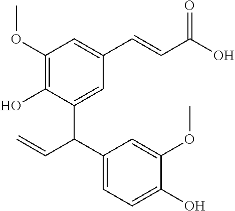 [SMILES: C=CC(c1ccc(O)c(OC)c1)c1cc(/C=C/C(=O)O)cc(OC)c1O] | Mobile phase: n-Hexane/EA = 53/47 Retention time: 13.2 min |

*Column used for HPLC was Hibar ® Fertigäute semi-preparation column (10 mm × 250 mm) using RI detector. The flow rates of the mobile phase were all 3.0 mL/min.

The physical properties and spectrum data of the compound I (3-Butylhexahydrophthalide) was listed below. Colorless oil. Formula: C₁₂H₂₀O₂. ESI-MS [M+H]⁺ m/z: 197.2. [α]_{D}²⁵−13.4° (c 0.13, CHCl₃). IR (neat) υₘₐₓ cm⁻¹: 2929, 2861, 1766, 1453 1369, 1128. ¹H-NMR (500 MHz, Chloroform-d) δ_{H}: 0.89 (3H, t, J=7.0 Hz), 1.20-1.76 (13H, m), 1.94 (1H, m), 2.16 (1H, m), 2.67 (1H, ₘ), 4.08 (1H, m). ¹³C-NMR (125 MHz, Chloroform-d) δc: 14.1, 22.7, 23.1, 23.3, 23.4, 27.5, 28.2, 33.1, 38.7, 39.6, 84.0, 178.7.

The physical properties and spectrum data of the compound II (Neocnidilide) was listed below. Colorless oil. Formula: C₁₂H₁₆O₂. ESI-MS [M+H]^{+ m/z:} 195.2. [α]_{D}²⁵−60.1° (c 0.37, CHCl₃). IR (neat) νₘₐₓ cm⁻¹: 2934, 2865, 1744, 1453, 1329, 1220, 1026. ¹H-NMR (500 MHz, Chloroform-d) δ_{H}: 0.89 (3H, t, J=7.0 Hz), 1.10-2.05 (10H, m), 2.17 (1H, m), 2.31 (1H, m), 2.46 (1H, m), 3.93 (1H, ddd, J=8.9, 7.6, 5.5 Hz), 6.74 (1H dd, J=6.7, 3.1 Hz). ¹³C-NMR (125 MHz, Chloroform-d) δ_{c}: 14.1, 20.9, 22.7, 25.2, 25.5, 27.7, 34.5, 43.2, 85.6, 131.3, 135.5, 170.5.

The physical properties and spectrum data of the compound III (5-[3-(4-Hydroxy-3-ethoxyphenyl)allyl]ferulic acid) was listed below Colorless viscid oil. Formula: C₂₀H₂₀O₆. ESI-MS [M-H]⁻m/z: 355.3. [α]_{D}²⁵+5.6° (c 0.12, CH₃OH). UV (MeOH) λₘₐₓ nm (log ε): 288 (3.9), 314 (3.8). IR (neat) νₘₐₓ cm⁻¹: 3444, 2935, 1633, 1509, 1434, 1376, 1269, 1153. ¹H-NMR (500 MHz, Acetone-d₆) δ_{H}: 3.77 (3H, s, 3′-OCH₃), 3.92 (3H, 3-OCH₃), 4.99 (1H, ddd, J=17.1, 1.8, 1.8 Hz, H-9′ₜᵣₐₙₛ), 5.10 (1H, br d, J=7.6 Hz, H-7′), 5.15 (1H, ddd, J=10.1, 1.8, 1.8 Hz, H-9′_{cis}), 6.33 (1H, d, J=15.9 Hz, H-8), 6.40 (1H, ddd, J=17.1, 10.1, 7.6 Hz, H-8′), 6.69 (1H, dd, J=7.9, 1.8 Hz, H-6′), 6.74 (1H, d, J=7.9 Hz, H-5′), 6.87 (1H, d, J=1.8 Hz, H-2′), 7.07 (1H, d, J=1.8 Hz, H-6), 7.22 (1H, d J=1.8 Hz, H-2), 7.56 (1H, d, J=15.9 Hz, H-7). ¹³C-NMR (125 MHz, Acetone-d₆) δ_{C}: 48.2 (C-7′), 56.4 (3′-OCH3), 56.6 (3-OCH3), 108.9 (C-2), 113.2 (C-2′), 115.6 (C-5′), 115.9 (C-9′) 116.1 (C-8), 121.8 (C-6′), 123.8 (C-6), 126.7 (C-1), 131.2 (C-5), 135.3 (C-1′), 141.5 (C-8′), 146.1 (C-4′), 146.2 (C-7), 147.2 (C-4), 148.2 (C-3′) 148.6 (C-3), 168.4 (C-9).

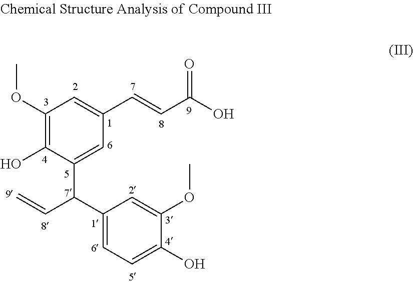 [SMILES: C=CC(c1ccc(O)c(OC)c1)c1cc(/C=C/C(=O)O)cc(OC)c1O]

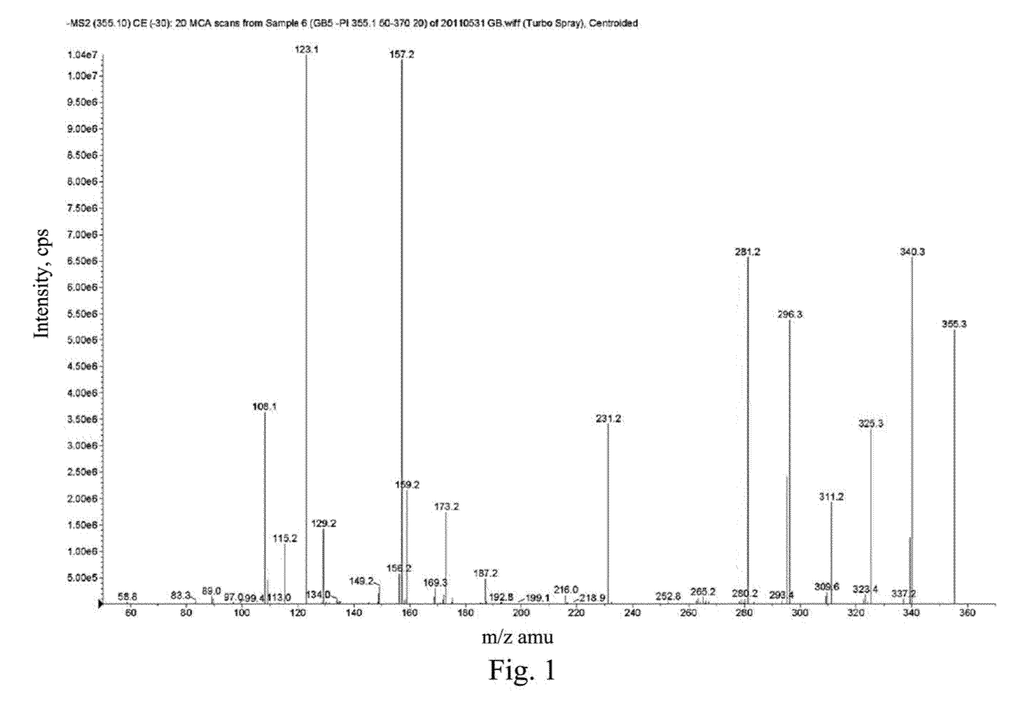

The obtained compound HI is colorless viscid oil. FIG. 1 is an ESI-MS spectrum of the compound III. From FIG. 1 it is observed that the measured m/z value of [M-H]⁻ was 355.3. Further from HR-ESI-MS, the measured m/z value of [M-H]⁻ was 355.1080, and the calculated m/z value of C₂₀H₂₀O₆ was 355.1182. Therefore, the chemical formula of the compound III was C₂₀H₂₀O₆, and the unsaturation degree of the compound III was 11.

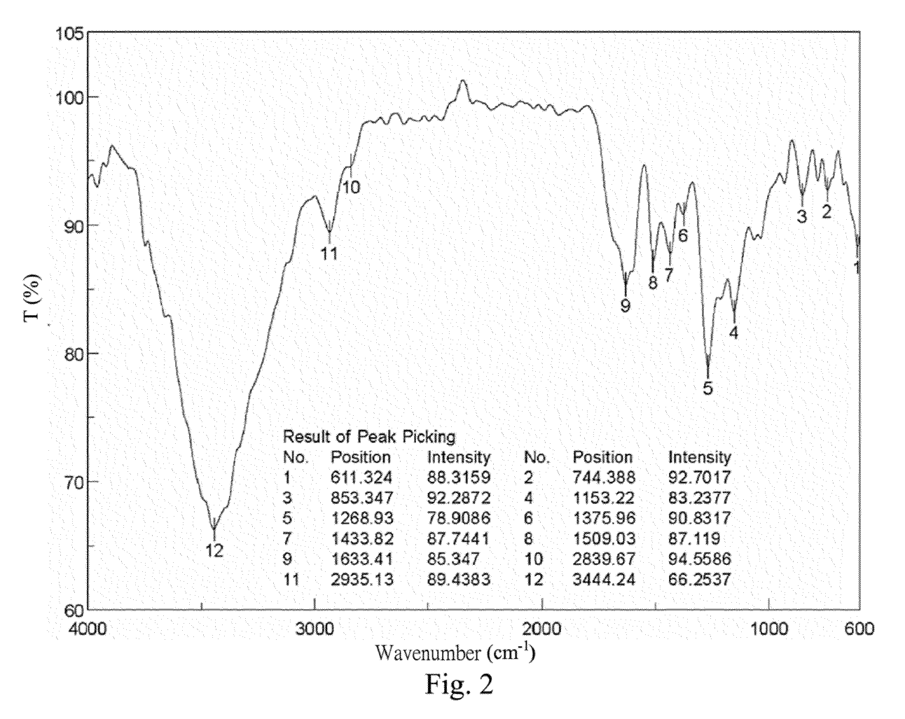

FIG. 2 is an IR spectrum of the compound III, The IR spectrum of the neat compound III has an O-H peak at 3444 cm⁻¹ (peak 12), a C═C peak at 1633 cm⁻¹ (peak 9), and a benzene C═C peak at 1509 cm⁻¹ (peak 8).

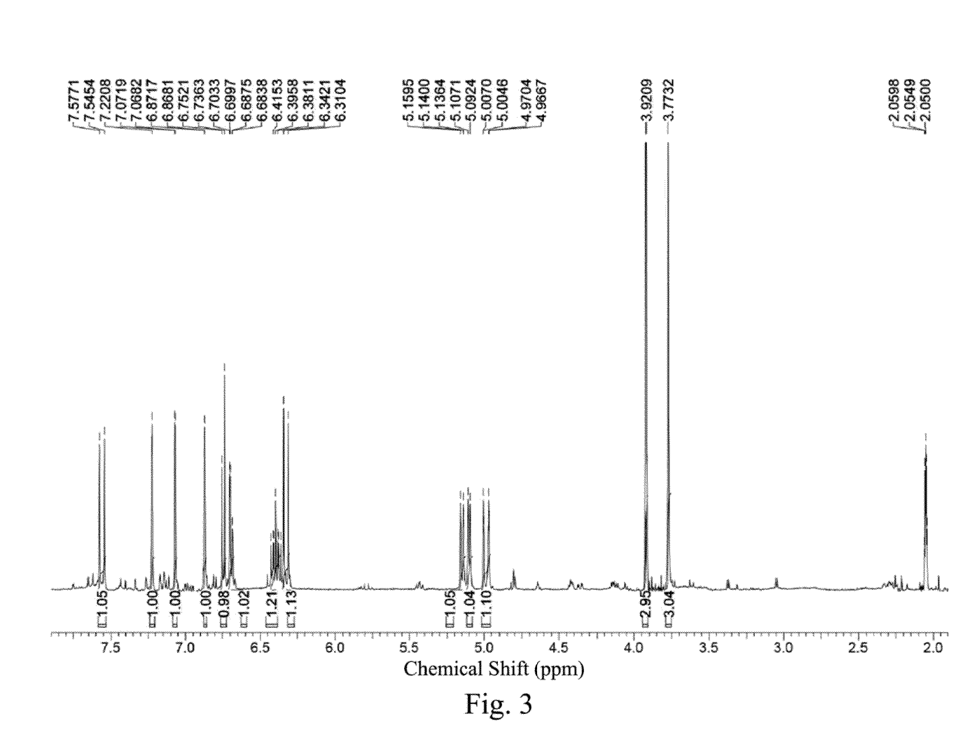

FIG. 3 is a ¹H NMR (500 MHz, acetone-do spectrum of the compound III. In FIG. 3, a set of signals located at δ_{H} 7.07 (1H, d, J=1.8 Hz) and 7.22 (1H, d, J=1.8 Hz) were belong to meta-coupling protons of a benzene ring. Therefore, a 4-substituted benzene ring might be presented in the compound III. A set of signals located at 3_{H} 6.69 (1H, dd, J=7.9, 1.8 Hz), δ_{H} 6.74 (1H, d, J=7.9 Hz), and 6.87 (1H, d, J=1.8 Hz) were belong to ABX coupling protons of a benzene ring. Therefore, a 3-substituted benzene ring might be presented in the compound III. A set of signals located at δ_{H} 6.33 (1H, d, J=15.9 Hz) and 7.56 (1H, d, J=15.9 Hz) were belong to trans protons of a double bond. A set of signals located at δ_{H} 4.99 (1H, ddd, J=17.1, 1.8, 1.8 Hz), 5.15 (1H, ddd, J=10.1, 1.8, 1.8 Hz) and 6.40 (1H, ddd, J=17.1, 10.1, 7.6 Hz) were belong to a terminal protons of a double bond. Moreover, the signal at δ_{H} 6.40 coupled to the signal at δ_{H} 5.10 (1H, br d, J=7.6 Hz), which was inferred to belong to a methine group. Therefore, a methine group connecting to a terminal double bond (i.e. —CH—CH═CH₂) might presented in the compound III. Hoverer, it was not sure that whether the signal at δ_{H} 5.10 was belonged to an oxymethine group, a double bond or other types of protons. A set of signals located at δ_{H} 3.77 (3H s) and δ_{H} 3.92 (3H, s) avere belonged to 2 methoxy (—OCH₃) group.

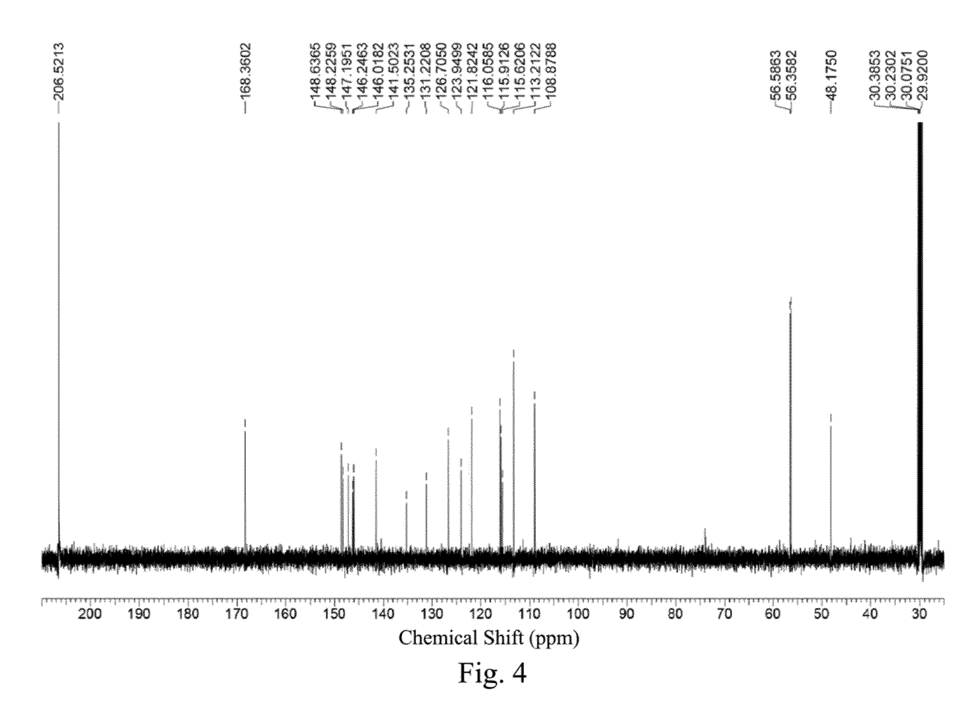

FIG. 4 is a ¹³C NMR (125 MHz, acetone-d₆) spectrum of the compound From ¹³C NMR and DEPT spectrum, it is observed that there were 8 quaternary carbons, 9 tertiary carbons (methine group), 1 secondary carbon (methylene group), and 2 primary carbons (methyl group). There were 20 carbons in total. A signal at δ_{C} 168.4 (s) was belonged to a carbonyl group of a carboxylic acid group. Sixteen signals at δ_{C} 108.9 (d), 113.2 (d), 115.6 (d), 115.9 (t), 116.1 (d), 121.8 (d), 123.8 (d), 126.7 (s), 131.2 (s), 135.3 (s), 141.5 (d), 146.1 (s), 146.2 (d), 147.2 (s), 148.2 (s), 148.6 (s) were belonged to two double bonds and two benzene rings, and one of the double bonds was a terminal double bonds. Signals at δ_{C} 56.4 (q) and 56.6 (q) were belonged to 2 methoxy groups. A signal at δ_{C} 48.2 (d) was belonged to a methine group, but not an oxymethine group or a double bond.

Since the chemical shifts difference (δ_{H} 6.33 and 7.56 of the trans double bond was large, together with the carbonyl group (δ_{C} 168.4) of the carboxylic acid group, and the multi-substituted benzene rings, methoxy groups, and disappeared hydroxyl groups, it was inferred that the compound III has a structure of a ferulic acid. That is, the compound III was a derivative of ferulic acid.

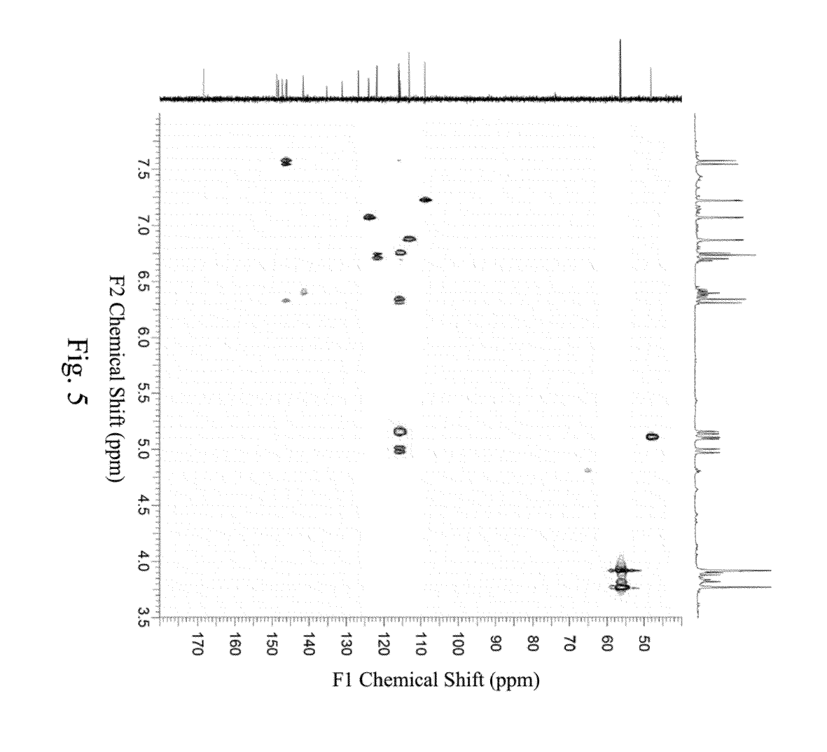

Next, HSQC and HMBC experiments were performed to determine the positions of each functional group. FIG. 5 is a HSQC (acetone-d₆) spectrum of the compound III. In FIG. 5, it can be seen that signals at δ_{H} 5.10 and δ_{C} 48.2 have cross peaks. Therefore, it can be sure that δ_{H} 5.10 was connected to δ_{C} 48.2.

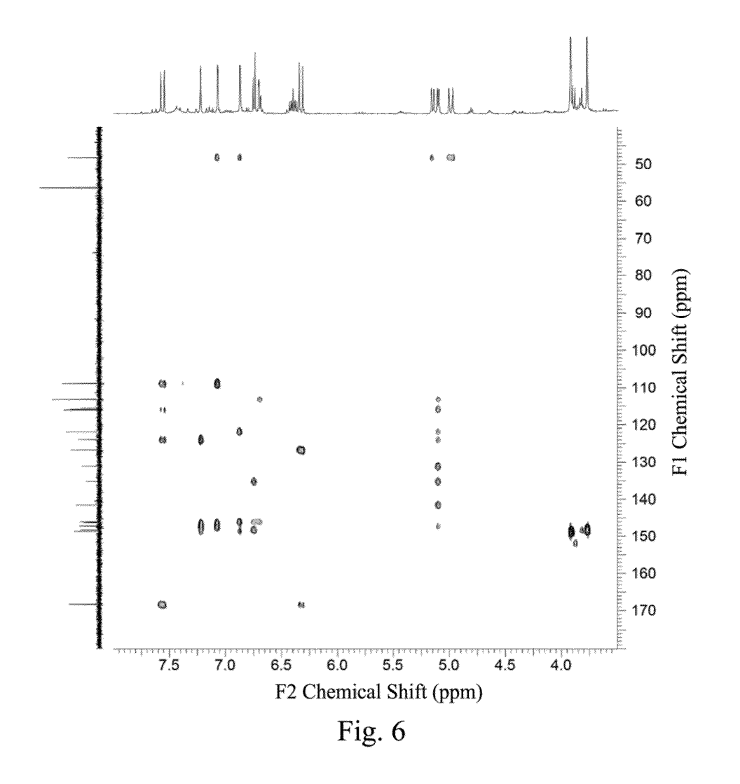

FIG. 6 is a HMBC (acetone-d₆) spectrum of the compound III. From FIG. 6, it can be observed that δ_{H} 5.10 was related to δ_{C} 113.2, 115.9, 121.8, 123.8, 131.2, 135.3, 141.5, and 147.2. Therefore, it can be sure that the methine group of δ_{H} 5.10 connected to 2 benzene rings and a terminal double bond. The position of the methine group of δ_{H} 5.10 was at C-7′, and the methine group of δ_{H} 5.10 connected to the carbons at positions C-5 and C-1′ of the two benzene rings, and the carbon at the position C-8′ of the terminal double bond. δ_{H} 7.56 was related to δ_{C} 108.9, 116.1, 123.8, 126.7, and 168.4. Therefore, it can be sure that a terminal of the trans double bond at positions C-7 and C-8 was connected to a carbon C-9 of a carboxylic acid group. The other terminal of the trans double bond at positions C-7 and C-8 was connected to a carbon C-1 of a benzene ring. δ_{H} 3.77 was related to δ_{C} 148.2, and δ_{H} 3.92 was related to δ_{C} 148.6. Therefore, it can be sure that the methoxy group of δ_{H} 3.77 was connected to C-3′, and the methoxy group of δ_{H} 3.92 was connected to C-3. The two methoxy groups above were respectively located at C-4 and C-4′.

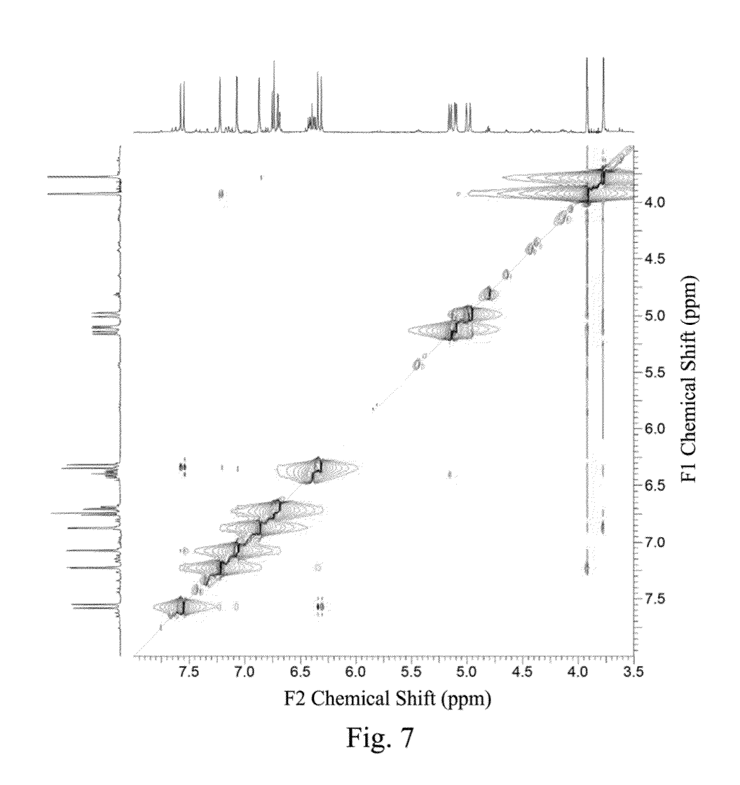

FIG. 7 is a NOESY (acetone-d₆) spectrum of the compound III. NOESY experiment was used to further confirm the positions of the methoxy groups. The result shows that δ_{H} 3.77 (3H, s) was related to δ_{H} 6.87 (1H, d, J=1.8 Hz). Therefore, it can be sure that the methoxy group of δ_{H} 3.77 was connected to C-3′, and the methoxy group of δ_{H} 3.92 was connected to C-3. The data about ¹³C NMR, DEPT, ¹H NMR, and HMBC were listed in the Table 3 below.

TABLE 3
NMR data of the compound III

| | ¹³C NMR* | ¹H NMR | HMBC |
|---|---|---|---|
| positions | δ_{C} (multi.) | δ_{H} (multi., J in Hz) | H→C |
| 1 | 126.7 (s) | | |
| 2 | 108.9 (d) | 7.22 (d, 1.6) | C-1, C-3, C-4, |
| | | | C-6 |
| 3 | 148.6 (s) | | |
| 4 | 147.2 (s) | | |
| 5 | 131.2 (s) | | |
| 6 | 123.8 (d) | 7.07 (d, 1.6) | C-2, C-4, C-7′ |
| 7 | 146.2 (d) | 7.56 (d, 15.9) | C-1, C-2, C-6, |
| | | | C-8, C-9 |
| 8 | 116.1 (d) | 6.33 (d, 15.9) | C-1, C-7, C-9 |
| 9 | 168.4 (s) | | |
| 1′ | 135.3 (s) | | |
| 2′ | 113.2 (d) | 6.87 (d, 1.8) | C-1′, C-3′, C-4′, |
| | | | C-6′, C-7′ |
| 3′ | 148.2 (s) | | |
| 4′ | 146.1 (s) | | |
| 5′ | 115.6 (d) | 6.74 (d, 7.9) | C-1′, C-3′, C-4′ |
| 6′ | 121.8 (d) | 6.69 (dd, 7.9, 1.8) | C-2′, C-4′, C-7′ |
| 7′ | 46.2 (d) | 5.10 (br d, 7.6) | C-4, C-5, C-6, |
| | | | C-1′, C-2′, C-6′, |
| | | | C-8′, C-9′ |
| 8′ | 141.5 (d) | 6.40 (ddd, 17.1, 10.1, 7.6) | C-5, C-1′, C-7′ |
| 9′ | 115.9 (t) | 4.99 (ddd, 17.1, 1.8, 1.8) | C-7′, C-8′ |
| | | 5.15 (ddd, 10.1, 1.8, 1.8) | C-7′ |
| 3-OCH₃ | 56.6 (q) | 3.92 (3H, s) | C-3 |
| 3′-OCH₃ | 56.4 (q) | 3.77 (3H, s) | C-3′ |

*The multiplicity of ¹³C NMR signals was determined by DEPT experiment.

From the data above, it can be sure that the compound III was 5-[3-(4-Hydroxy-3- thoxyphenyl)allyl]ferulic acid.

### Assay of Tyrosinase Activity

The source of the tested tyrosinase was from mushroom, and the substrate of the tyrosinase was L-tyrosine. The experiment was performed by the method below. First, 40 μL of pH 6.8 phosphate buffer solution, 40 μL of various sample solution (samples in 0.07M pH6.8 phosphate buffer solution containing 0.1 vol % of DMSO), and 40 μL of tyrosinase (250 unit/mL, dissolved in pH 6.8 phosphate buffer solution at a concentration of 0.07 M) were sequentially and respectively added into 96-wells plate. The sample solutions above respectively contain the compounds II, III, and kojic acid. The group added with the sample solution containing kojic acid was used as positive control (PC). A group without adding the sample solution was used as control (C).

The mixtures described above were thermostat at 37° C. for 10 minutes. Then, 80 μL of tyrosine solutions (0.4 mg/mL, dissolved in 0.07M pH6.8 phosphate buffer solution) were respectively added into each mixture and thermostat for another 20 minutes to perform reaction. The amount of the obtained product L-DOPA of each mixture was measured at 475 nm to get absorbance of each sample. Therefore, the tyrosinase activity was calculated by the formula below.

Tyrosinase activity(%)=[(A−B)/(C−D)]×100

The symbol A above is the absorbance of groups containing a sample and tyrosinase. The symbol B above is the absorbance of groups containing a sample but not tyrosinase. The symbol C above is the absorbance of groups containing tyrosinase but not a sample. The symbol D above is the absorbance of groups does not contain both a sample and tyrosinase.

The results of the assay showed that the compounds I, II, and III do not show obvious inhibitory effect to the tyrosinase activity.

### Viability Test of B16-F10 Murine Melanoma Cells

In this test, B16-F10 murine melanoma cells (BCRC No. 60031) were used.

First, the B16-F10 murine melanoma cells were cultured in Dulbecco's Modified Eagle's Medium (abbreviated as DMEM, which contains 90% (v/v) DMEM, 10% (v/v) fetal bovine serum), and additionally added 1% (v/v) Penicillin-Streptomycin Solution. Then, the cells in the mediums were incubated under 5% CO₂/air at 37° C. in 70% of humidity. The medium was exchanged every two days. When the cells grew to a density of 80-90%, the cells were treated by trypsin to desorb the cells from the walls of containers Next, haemocytometer (Neubauer Improved., Marienfeld, Germany) was used to count the number of the cells. Proper amount of cells were then transplanted into cell culture plates to prepare for cell viability tests.

Cell viability rate was determined by MTT assay. MTT assay was performed by using lactate dehydrogenase in the mitochondria of living cells to reduce yellow 3-(4,5-dimethylthiazol-2-yl)-2,5-diphenyl tetrazolium bromide (MTT) to purple formazan product. Therefore, the amount of the purple formazan product is proportional to the number of the living cells and thus can be determined by spectrophotomter.

In a 12-wells cell plate, 1×10³ cells were implanted into each well and stayed for 24 hours to let the cells attach to the wail of the well. Then, the compounds I, II, Ill and arbutin (positive control; PC) were respectively added into each wells and cultured for 72 hours. Next, MTT dye (Applichem Co., Denmark) dissolved in 0.5 mg/ml PBS solution was added to each well to react for 2 hours. The obtained purple formazan product was dissolved in 400 μL of DMSO, and then the absorbance of the purple DMSO solution was analyzed by microplate reader to get the absorbance at 570 nm. The cell viability rate was determined by the formula below.

Cell viability rate (%)=(Absorbance of test group/absorbance of blank group)×100%

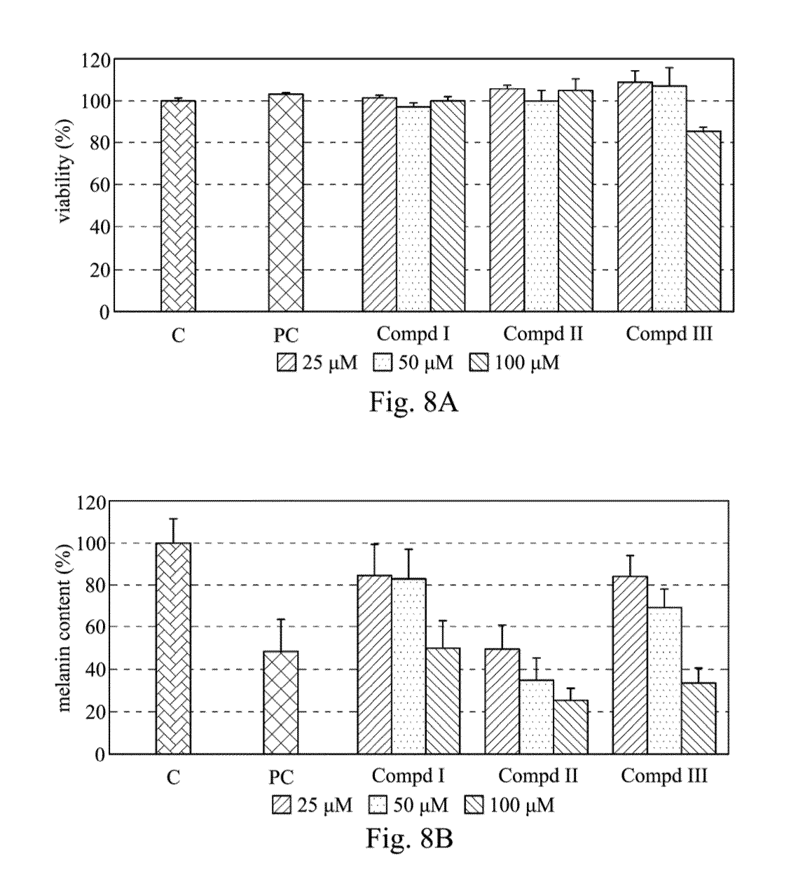

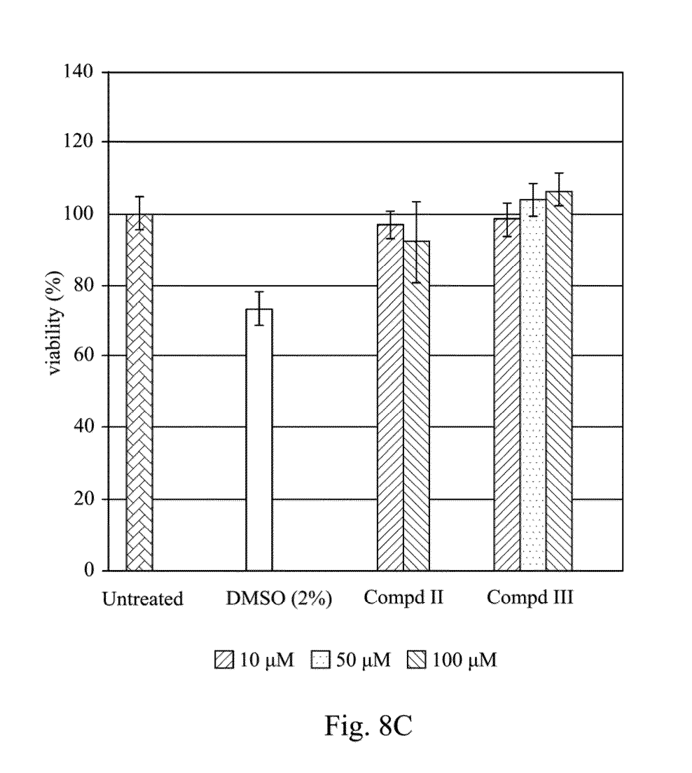

FIG. 8A is an experimental result of the cell viability of B16-F10 murine melanoma cells. From FIG. 8A, it is observed that compounds I and II did not affect the cell viability, and the compound III had a slight tendency to inhibit cell growth at a concentration of 100 μM. Basically, compounds I, II and III did not have cytotoxicity to the B16-F10 murine melanoma cells.

### Analysis of Melanin Content in B16-F10 Murine Melanoma Cells

The melanin content in B16-F10 murine melanoma cells was analyzed by a modified Hosoi method disclosed in 1985 (Hosoi J, Abe E, Suda T, Kuroki T, Regulation of melanin synthesis of B16 mouse melanoma cells by 1 alpha, 25-dihydroxyvitamin D3 and retinoic acid. Cancer Res. 1985; 45(4):1474-8).

First, in a 12-wells cell plate, 1×10³ cells were implanted into each well and stayed for 24 hours to let the cells attach to the wall of the well. Various concentrations (25, 50, and 100 μM) of the compounds I, II, III, and arbutin (positive control; PC) were respectively added into each wells, and 100 nM of α-melanocyte stimulating hormone (α-MSH) was subsequently adaded. The cells were cultured for 72 hours. After the treatment above, cells were washed by PBS, and then treated by trypsin to desorb the cells. 150 μL of 1N NaOH solution containing 10% DMSO was then added and heated in a 80° C. water bath for 1 hour. After cooling to room temperature, the samples were centrifuge at 5000 rpm for 10 minutes. The upper solution was transferred into a 96-wells plate and analyzed by a microplate reader at 405 nm. The absorbance at 405 nm for each sample was read to calculate the melanin content of each sample.

FIG. 8B is an experimental result of the melanin content in B16-F10 murine melanoma cells. From FIG. 8B, it is observed that compounds I, II and III can decrease the melanin content in the melanoma cells without affecting the cell viability rate. The IC₅₀ of the compounds I, II and III for inhibiting the melanogenesis were 100.1±12.9 μM, 31.1±6.8 μM, and 78.9±8.1 μM, respectively. Therefore, it is observed that the compound II has the best inhibitory activity for melanogenesis.

### Viability Test of Human Epidermal Skin Equivalents

In this test, fresh mamma reduction surplus skin of a single female individual (D002, 59 years, Fitzpatrick skin type I/II) was used for isolation of normal human epidermal keratinocytes (NHEKs).

Leiden epidermal models (LEMs) were generated by seeding NHEKs on an inert filter insert. The NHEKs in LEMS were incubated overnight under submerged conditions in keratinocyte medium. Within four days, fetal bovine serum was gradually omitted and the NHEKs in LEMs were cultured serum-free at the air-liquid interface for 7 days, while culture medium was refreshed twice a week.

Viability assays were performed by adding 0,5 mL of 1 mg/mL MTT to each of the NHEKs in LEMs for 3h, after 24 hours exposure to the test articles (compounds II, III, and DMSO (negative control)). The precipitated blue formazan product was extracted from the cells within 2 hours with 0.5 mL isopropanol per well. The concentration of formazan was measured by determining the OD at 570 nm using a Tecan Infinite F50 microplate reader.

FIG. 8C is an experimental result of the cell viability of the NHEKs in Leiden epidermal models. From FIG. 8C, it is observed that compounds II and III did not affect the cell viability at a concentration of 10-100 μM. The cell viability of compounds II is 97% (10 μM), and 92% (100 μM). The cell viability of compounds Ill is 98% (10 μM), 104 (50 μM), and 106% (100 μM). Accordingly, compounds II and Ill did not have cytotoxicity to the NHEKs in the Leiden epidermal models.

All the features disclosed in this specification (including any accompanying claims, abstract, and drawings) may be replaced by alternative features serving the same, equivalent or similar purpose, unless expressly stated otherwise. Thus, each feature disclosed is one example only of generic series of equivalent or similar features.

What is claimed is:

1. An isolated compound III having a chemical structure as follow:

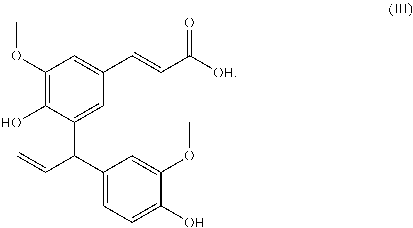 [SMILES: C=CC(c1ccc(O)c(OC)c1)c1cc(/C=C/C(=O)O)cc(OC)c1O]

2. A method of inhibiting melanogenesis, the method comprises:
applying a composition comprising an effective amount of a compound having at least one of the chemical structures shown below on skin to inhibit melanogenesis. 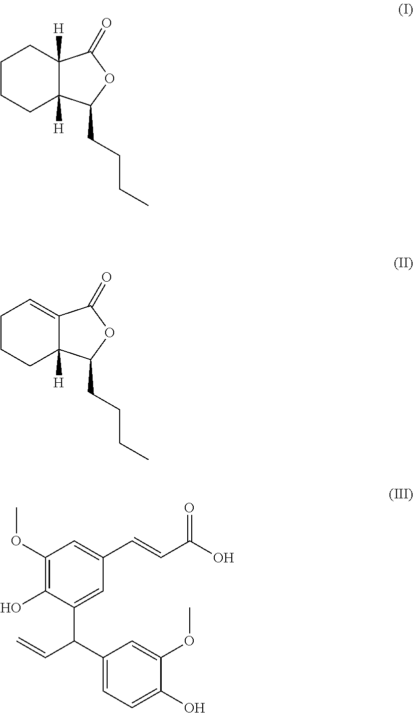 [SMILES: CCCC[C@@H]1OC(=O)[C@H]2CCCC[C@@H]12 ; CCCC[C@@H]1OC(=O)C2=CCCC[C@H]21 ; C=CC(c1ccc(O)c(OC)c1)c1cc(/C=C/C(=O)O)cc(OC)c1O]
